(19) United States
(12) Patent Application Publication
(10) Pub. No.: US 2008/0020054 A1
(43) Pub. Date: Jan. 24, 2008

(54) Cosmetic and Dermatological Preparations Containing Transparent Surface-Coated Titanium Dioxide Particles

(75) Inventors: Valerie Andre, Ludwigshafen (DE); Scott D Thomson, Sparta, NJ (US)

Correspondence Address: CONNOLLY BOVE LODGE & HUTZ LLP, 1875 EYE STREET, N.W., SUITE 1100, WASHINGTON, DC, 20036, US

(73) Assignee: Basf Aktiengesellschaft, Ludwigshafen (DE)

(21) Appl. No.: 11/817,619

(22) PCT Filed: Mar. 3, 2006
(86) PCT No.: PCT/EP06/60451

§ 371 Date: Aug. 31, 2007

(30) Foreign Application Priority Data
Mar. 7, 2005 (DE) 102005010803.2

(51) Int. Cl. A61K 8/29; A61K 33/00; A61K 9/14; A61Q 19/00; A61Q 5/00; A61Q 17/04; A61K 33/24

(52) U.S. Cl. 424/490; 424/59; 424/617; 424/70.1

(57) ABSTRACT

The present invention relates to cosmetic and dermatological preparations for protecting human skin and hair against UV radiation, comprising surface-coated titanium dioxide particles which are transparent in the visible region and have a crystallite size of from 10 to 20 nm and a specific surface area of from 90 to 110 m²/g, wherein the surface coating of the titanium dioxide particles comprises a multicoating of a) aluminum oxide and b) methicone or a copolymer of methicone and dimethicone or a multicoating of a) aluminum oxide, b) methicone or a copolymer of methicone and dimethicone and c) silicon dioxide.

The present invention relates to cosmetic and dermatological preparations for protecting human skin and hair against UV radiation, comprising surface-coated titanium dioxide particles which are transparent in the visible region and have a crystallite size of from 10 to 20 nm and a specific surface area of from 90 to 110 m²/g, wherein the surface coating of the titanium dioxide particles comprises a multicoating of a) aluminum oxide and b) methicone or a copolymer of methicone and dimethicone or a multicoating of a) aluminum oxide, b) methicone or a copolymer of methicone and dimethicone and c) silicon dioxide.

Metal oxides such as titanium dioxide or zinc oxide are widespread in sunscreen compositions. Their effect is based essentially on reflection, scattering and absorption of the harmful UV radiation and is essentially dependent on the primary particle size of the metal oxides.

Microfine titanium dioxide is used widely in cosmetic formulations since it is chemically stable and toxicologically safe and leads neither to skin irritations nor to sensitization. It is currently the most often used and most important mineral photoprotective substance.

A disadvantage of metal oxides such as titanium dioxide or zinc oxide is their photocatalytic activity, which triggers reactions which can lead to the formation of reactive species (e.g. hydroxyl radicals).

The prior art attempts to reduce the photocatalytic activity of these metal oxides without reducing the UV-screening properties by covering them, for example, with a coating of inorganic and/or organic surface components. These surface components may, inter alia, be Al₂O₃, SiO₂ and/or siloxanes.

EP-A-0 988 853 describes metal oxide particles coated with silicon dioxide, and their preparation and use as constituent in sunscreen compositions. A disadvantage here is that these coated metal oxide particles have low surface functionality and a high degree of particle intergrowth. This firstly hinders the incorporation of the particles into a cosmetic formulation, and secondly restricts their stability with regard to sedimentation.

EP 0 869 762 B1 describes silicone polymer coated hydrophobicized metal oxides and their use as sunscreens in cosmetic products.

In addition, EP 1 284 277 A1 describes metal oxide particles coated with silicon dioxide and having a small structure, and sunscreens prepared therewith.

Titanium dioxide nanoparticles with particle sizes below about 30 nm are potentially suitable for use as UV absorbers in cosmetic and dermatological preparations.

Particles, particle aggregates or particle agglomerates of titanium dioxide which are larger than about 30 nm lead to scattered-light effects and thus to an undesired decrease in transparency in the visible light region. For this reason, the redispersibility, i.e. the ability of the prepared titanium dioxide nanoparticles to be converted to a colloidally disperse state, is an important prerequisite for the abovementioned applications.

On account of the size quantization effect, titanium dioxide nanoparticles with particle sizes below about 5 nm exhibit a blue shift of the absorption edge (L. Brus, J. Phys. Chem. (1986), 90, 2555-2560) and are therefore less suitable for use as UV absorbers in the UV-A region.

As the primary particle size decreases, the specific surface area increases and thus the active surface for the formation of aggregates and agglomerates, and also for adsorption processes, through which the stability of emulsions can be endangered.

However, agglomerates are undesired in cosmetic transparent formulations since they are discernible as particles on the skin, often even with the naked eye, reduce the transparency and also the UV protective effect of a sunscreen and settle out during storage. Agglomerates can also lead to instability of the formulation.

In practice, it has therefore been found that as the finely divided nature of the particles grows, the dispersion problems increase, meaning that the dispersion and stabilization process overall represents one of the most complex stages during the manufacture of cosmetic formulations.

It was thus the object of the present invention to provide cosmetic and dermatological photoprotective agent preparations which comprise titanium dioxide particles and with which the disadvantages of the prior art can be overcome. In particular, the titanium dioxide particles should have low photocatalytic activity and low scattering behavior. In addition, the titanium dioxide particles should be transparent in the visible region and be easy to incorporate into cosmetic dispersions. If possible, the particles should not undergo irreversible aggregation so that a complex dispersion process can be avoided.

This object has now been achieved through the provision of cosmetic and dermatological preparations for protecting human skin and hair against UV radiation, comprising surface-coated titanium dioxide particles which are transparent in the visible region and have a crystallite size of from 10 to 20 nm and a specific surface area of from 90 to 110 m²/g, wherein the surface coating of the titanium dioxide particles comprises a multicoating of a) aluminum oxide and b) methicone or a copolymer of methicone and dimethicone.

The designation methicone and dimethicone stands for methyl hydrogen polysiloxane and dimethyl polysiloxane, respectively.

The crystallite size of the surface-coated titanium dioxide particles present according to the invention in the cosmetic and dermatological preparations is in the range from 10 to 20 nm, preferably 12 to 18 nm, particularly preferably 14 to 16 nm, determined by means of X-ray diffraction spectroscopy.

The BET surface area, determined in accordance with DIN 66131 [see also F. M. Nelsen, F. T. Eggertsen, Analyt. Chem. 30 (1958), [387], of the surface-coated titanium dioxide particles used according to the invention in the cosmetic and dermatological preparations is in a range between 90 and 110 m²/g, preferably between 95 and 105 m²/g.

A preferred form of the cosmetic and dermatological preparation is one in which the surface coating of the titanium dioxide particles comprises silicon dioxide as additional component c).

A particularly preferred form of the cosmetic and dermatological preparation is one in which the titanium dioxide particles are coated by
a) a first layer of aluminum oxide or a mixture of aluminum oxide and silicon dioxide and
b) an outer layer of a copolymer of methicone and dimethicone (CAS No: 68037-59-2).

The thickness of the multicoating of aluminum oxide, silicon dioxide and methicone or the copolymer of methicone and dimethicone can be varied here between 0.5 and 3 nm. Within this range, the particles display adequately high UV-absorption, reflection and scattering.

The surface-coated titanium dioxide particles used according to the invention are obtainable, for example, by a method described in U.S. Pat. No. 6,660,380 for coated zinc oxide particles.

A further preferred embodiment of the cosmetic and dermatological preparations is one in which the transparent surface-coated titanium dioxide particles have a titanium dioxide content of from 70 to 92% by weight, preferably 72 to 90% by weight, particularly preferably 73 to 83% by weight, where the percentages refer to the total weight of the surface-coated titanium dioxide particles present in the cosmetic and dermatological preparations.

A likewise preferred embodiment of the cosmetic and dermatological preparations is one in which the transparent surface-coated titanium dioxide particles have a silicon dioxide content of from 4 to 10% by weight, preferably 5 to 9% by weight, particularly preferably 6.5 to 8.5% by weight, where the percentages refer to the total weight of the surface-coated titanium dioxide particles present in the cosmetic and dermatological preparations.

A further preferred embodiment of the cosmetic and dermatological preparations is one in which the transparent surface-coated titanium dioxide particles have an aluminum oxide content of from 1 to 10% by weight, preferably 1 to 5% by weight, particularly preferably 2.5 to 4.5% by weight, where the percentages refer to the total weight of the surface-coated titanium dioxide particles present in the cosmetic and dermatological preparations.

A further preferred embodiment of the cosmetic and dermatological preparations is one in which the transparent surface-coated titanium dioxide particles have a methicone or methicone/dimethicone copolymer content of from 3 to 10% by weight, preferably 4 to 7% by weight, particularly preferably 4.5 to 6.5% by weight, where the percentages refer to the total weight of the surface-coated titanium dioxide particles present in the cosmetic and dermatological preparations.

A further preferred embodiment of the cosmetic and dermatological preparations is one in which the titanium dioxide particles have a rutile fraction of from 94 to 100%, preferably 98 to 100%, particularly preferably from 98.5 to 99.5%.

The content of surface-coated titanium dioxide particles which are transparent in the visible region in the cosmetic or dermatological preparations according to the invention is in the range from 0.1 to 25% by weight, preferably 0.1 to 10% by weight, particularly preferably 1 to 7% by weight, based on the total weight of the cosmetic and dermatological preparations.

The cosmetic and pharmaceutical preparations comprising photoprotective agents are generally based on a carrier which comprises at least one oil phase. However, merely aqueous-based preparations are also possible. Accordingly, oils, oil-in-water and water-in-oil emulsions, creams and pastes, lip protective stick masses or gels are suitable.

Suitable emulsions are, inter alia, also O/W macroemulsions, O/W microemulsions or O/W/O emulsions with surface-coated titanium dioxide particles present in dispersed form, the emulsions being obtainable by phase inversion technology, as in DE-A-197 26 121.

Customary cosmetic auxiliaries which may be suitable as additives are, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators.

Suitable coemulsifiers are preferably known W/O and also O/w emulsifiers, such as, for example, polyglycerol esters, sorbitan esters or partially esterified glycerides. Typical examples of fats are glycerides; waxes to be mentioned are, inter alia, beeswax, paraffin wax or microcrystalline waxes, if appropriate in combination with hydrophilic waxes.

Stabilizers which can be used are metal salts of fatty acids, such as, for example, magnesium stearate, aluminum stearate and/or zinc stearate.

Suitable thickeners are, for example, crosslinked polyacrylic acids and derivatives thereof, polysaccharides, in particular xanthan gum, guar, agar, alginates and tyloses, carboxymethylcellulose and hydroxyethylcellulose, also fatty alcohols, monoglycerides and fatty acids, polyacrylates, polyvinyl alcohol and polyvinyl pyrrolidone.

Biogenic active ingredients are understood as meaning, for example, plant extracts, protein hydrolyzates and vitamin complexes.

Customary film formers are, for example, hydrocolloids, such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds.

Suitable preservatives are, for example, formaldehyde solution, p-hydroxybenzoate or sorbic acid.

Suitable pearlizing agents are, for example, glycol distearic esters, such as ethylene glycol distearate, but also fatty acids and fatty acid monoglycol esters.

Dyes which can be used are the substances approved and suitable for cosmetic purposes, as are listed, for example, in the publication “Kosmetische Farbemittel” [Cosmetic Colorants] from the Farbstoffkommission der Deutschen Forschungsgemeinschaft [Dyes Commission of the German Research Society], published by Verlag Chemie, Weinheim, 1984. These dyes are usually used in concentrations of from 0.001 to 0.1% by weight, based on the total mixture.

An additional content of antioxidants is generally preferred. Thus, favorable antioxidants which may be used are all antioxidants which are customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thiorodoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. sodium ascorbate, ascorbyl palmitate, Mg ascorbyl phosphate, ascorbyl acetate), tocopherol and derivatives (e.g. vitamin E acetate, tocotrienol), vitamin A and derivatives (vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are the antioxidant or the antioxidants, it is advantageous to choose their particular concentration from the range from 0.001 to 10% by weight, based on the total weight of the formulation.

If vitamin A and/or derivatives thereof or carotenoids are the antioxidant or the antioxidants, it is advantageous to choose their particular concentration from the range from 0.001 to 10% by weight, based on the total weight of the formulation.

If the cosmetic or dermatological preparation for the purposes of the present invention is a solution or emulsion or dispersion, solvents which can be used are:

water or aqueous solutions; oils, such as triglycerides of capric acid or of caprylic acid, but preferably castor oil; fats, waxes and other natural and synthetic fatty bodies, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl and monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water may be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, from the group of esters of aromatic carboxyic acids and saturated and/or unsaturated, branched and/or unbranched alcohols of chain length from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids of chain length from 8 to 24, in particular 12-18, carbon atoms.

The fatty acid triglycerides can, for example, be chosen advantageously from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. It may also if appropriate be advantageous to use waxes, for example cetyl palmitate, as the sole lipid component of the oil phase.

The oil phase is advantageously chosen from the group consisting of 2-ethylhexyl isostearate, isohexadecane, octyldodecanol, isotridecyl isononanoate, isoeicosane, 2-ethylhexyl cocoate, C₁₂-C₁₅-alkyl benzoate, caprylic/capric triglyceride, dicaprylyl ether.

Mixtures of C₁₂-C₁₅-alkyl benzoate and 2-ethylhexyl isostearate, mixtures of C₁₂-C₁₅-alkyl benzoate and isotridecyl isononanoate, and mixtures of C₁₂-C₁₅-alkyl benzoate, 2-ethylhexyl isostearate and isotridecyl isononanoate are particularly advantageous.

Of the hydrocarbons, paraffin oil, squalane and squalene are to be used advantageously for the purposes of the present invention.

Advantageous oil components are also, for example, butyloctyl salicylate (for example that available under the trade name Hallbrite BHB from CP Hall), hexadecyl benzoate and butyloctyl benzoate and mixtures thereof (Hallstar AB) and/or diethylhexyl naphthalate (Hallbrite TQ).

The oil phase can also advantageously have a content of cyclic or linear silicone oils or consist entirely of such oils, although it is preferred to use an additional content of other oil phase components apart from the silicone oil or the silicone oils.

Cyclomethicone (octamethylcyclotetrasiloxane) is advantageously used as silicone oil to be used according to the invention. However, other silicone oils are also to be used advantageously for the purposes of the present invention, for example hexamethylcyclotrisiloxane, polydimethylsiloxane, poly(methylphenylsiloxane).

In addition, mixtures of cyclomethicone and isotridecyl isononanoate, and of cyclomethicone and 2-ethylhexyl isostearate are particularly advantageous.

Gels used according to the invention usually comprise alcohols of low carbon number, e.g. ethanol, isopropanol, 1,2-propanediol, glycerol and water or an abovementioned oil in the presence of a thickener, which in the case of oily-alcoholic gels is preferably silicon dioxide or an aluminum silicate, and in the case of aqueous-alcoholic or alcoholic gels is preferably a polyacrylate.

Solid sticks comprise, for example, natural or synthetic waxes, fatty alcohols or fatty acid esters. Preference is given to using lipcare sticks, and stick formulations for body deodorization.

Suitable base materials which are suitable for use as cosmetic sticks for the purposes of the present invention are liquid oils (e.g. paraffin oils, castor oil, isopropyl myristate), semisolid constituents (e.g. Vaseline, lanolin), solid constituents (e.g. beeswax, ceresine and microcrystalline waxes and ozokerite), and high-melting waxes (e.g. carnauba wax, candelilla wax).

For the purposes of the present invention, suitable propellants for cosmetic and/or dermatological preparations which can be sprayed from aerosol containers are the customary known readily volatile, liquefied propellants, for example hydrocarbons (propane, butane, isobutane), which can be used on their own or in a mixture with one another. Compressed air is also used advantageously.

The person skilled in the art is of course aware that there are propellant gases which are per se nontoxic which would in principle be suitable for realizing the present invention in the form of aerosol preparations but which nevertheless should be avoided due to a harmful effect on the environment or other accompanying circumstances, in particular fluorinated hydrocarbons and chlorofluorocarbons (CFCs).

Cosmetic preparations for the purposes of the present invention can also be in the form of gels which, besides an effective content of the active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel e.g. in an amount between 0.1 and 30% by weight, preferably between 0.5 and 15% by weight.

The total fraction of the auxiliaries and additives can be 1 to 80% by weight, preferably 6 to 40% by weight and the nonaqueous fraction (“active substance”) is 20 to 80% by weight, preferably 30 to 70% by weight, based on the compositions. The compositions can be prepared in a manner known per se, i.e. for example by hot, cold, hot-hot/cold or PIT emulsification. This is a purely mechanical process, a chemical reaction does not take place.

The sunscreen preparations according to the invention can accordingly be present in liquid, paste or solid form, for example as water-in-oil creams, oil-in-water creams and lotions, aerosol foam creams, gels, oils, fatty sticks, powders, sprays or alcohol-aqueous lotions.

Finally, further substances which absorb in the UV region and are known per se can be co-used provided they are stable in the overall system of the combination of UV filters to be used according to the invention.

The majority of the photoprotective agents in the cosmetic and pharmaceutical preparations which serve to protect the human epidermis consists of compounds which absorb UV light in the UV-B region, i.e. in the region from 280 to 320 nm. For example, the fraction of the UV-A absorbers to be used according to the invention is 10 to 90% by weight, preferably 20 to 50% by weight, based on the total amount of UV-B and UV-A absorbing substances.

Suitable UV filter substances which are used in combination with the transparent surface-coated titanium dioxide particles to be used according to the invention are any UV-A and UV-B filter substances. Examples are:

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2- | 52793-97-2 |
| | one methyl sulfate | |
| 3 | 3,3,5-Trimethylcyclohexyl salicylate | 118-56-9 |
| | (homosalatum) | |
| 4 | 2-Hydroxy-4-methoxybenzophenone | 131-57-7 |
| | (oxybenzonum) | |
| 5 | 2-Phenylbenzimidazole-5-sulfonic acid and its | 27503-81-7 |
| | potassium, sodium and triethanolamine salts | |
| 6 | 3,3′-(1,4-Phenylenedimethine)bis(7,7-dimethyl- | 90457-82-2 |
| | 2-oxobicyclo[2.2.1]heptane-1- | |
| | methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzonum) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl-o-aminobenzoate or: | 134-09-8 |
| | 5-methyl-2-(1-methylethyl) 2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone | 131-53-3 |
| | (dioxybenzone) | |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxalic acidic sodium | |
| 26 | 3-(4′-Sulfobenzylidene)bornan-2-one and its | 56039-58-8 |
| | salts | |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)- | 103597-45-1 |
| | 4-(1,1,3,3-tetramethylbutyl)phenol] | |
| 30 | 2,2′-(1,4-Phenylene)bis-1H-benzimidazole- | 180898-37-7 |
| | 4,6-disulfonic acid, Na salt | |
| 31 | 2,4-bis[4-(2-Ethylhexyloxy)-2-hydroxy]phenyl- | 187393-00-6 |
| | 6-(4-methoxyphenyl)(1,3,5)-triazine | |
| 32 | 3-(4-Methylbenzylidene)camphor | 36861-47-9 |
| 33 | Polyethoxyethyl 4- | 113010-52-9 |
| | bis(polyethoxy)paraaminobenzoate | |
| 34 | 2,4-Dihydroxybenzophenone | 131-56-6 |
| 35 | 2,2′-Dihydroxy-4,4′-dimethoxybenzophenone- | 3121-60-6 |
| | 5,5′-disodium sufonate | |
| 36 | Benzole acid, 2-[4-(diethylamino)-2- | 302776-68-7 |
| | hydroxybenzoyl], hexyl ester | |
| 37 | 2-(2H-Benzotriazol-2-yl)-4-methyl-6-[2- | 155633-54-8 |
| | methyl-3-[1,3,3,3-tetramethyl-1- | |
| | [(trimethylsilyl)oxy]disiloxanyl]propyl]phenol | |
| 38 | 1,1-[(2,2′-Dimethylpropoxy)carbonyl]- | 363602-15-7 |
| | 4,4-diphenyl-1,3-butadiene | |

Polymeric or polymer-bound filter substances can also be used according to the invention.

The cosmetic and dermatological preparations according to the invention can advantageously also comprise inorganic pigments based on metal oxides and/or other metal compounds which are insoluble or sparingly soluble in water, chosen from the group of oxides of zinc (ZnO), iron (e.g. Fe₂O₃), zirconium (ZrO₂), silicon (SiO₂), manganese (e.g. MnO), aluminum (Al₂O₃), cerium (e.g. Qe₂O₃), mixed oxides of the corresponding metals, and mixtures of such oxides.

Particular preference is given to pigments based on ZnO.

The inorganic pigments may be present here in coated form, i.e. they are surface-treated. This surface treatment can, for example, consist in providing the pigments with a thin hydrophobic layer by a method known per se, as described in DE-A-33 14 742.

To protect human hair against UV rays, the photoprotective agent formulations according to the invention can be incorporated into shampoos, lotions, gels, hair sprays, hair colorants, aerosol foam creams or emulsions in concentrations of from 0.1 to 10% by weight, preferably 1 to 7% by weight. The formulations in each case can be used here, inter alia, for the washing, coloring and the styling of hair.

The formulations to be used according to the invention are generally characterized by a particularly high absorption capacity in the region of UV-A radiation with a sharp band structure. In addition, they are readily soluble in cosmetic oils and can be incorporated easily into cosmetic formulations. The emulsions prepared with the formulations are characterized in particular by their high stability, and the preparations prepared therewith are characterized by their pleasant feel on the skin.

The UV filter effect of the formulations according to the invention can also be exploited for stabilizing active ingredients and auxiliaries in cosmetic and pharmaceutical formulations.

By reference to the examples below, the aim is to illustrate the subject-matter of the present invention in more detail.

### EXAMPLE 1

| Phase | % | INCI |
|---|---|---|
| A | 3.0 | Laurylmethicone Copolyol |
| | 6.25 | Titanium Dioxide, Silica Hydrate, Alumina |
| | | Hydrate, Methicone/Dimethicone Copolymer |
| | 11.25 | Mineral Oil |
| | 3.0 | C₁₂-C₁₅ Alkyl Benzoate |
| | 2.0 | Isopropyl Myristate |
| | 2.0 | C₃₀-C₄₅ Alkyl Methicone |
| B | ad 100 | Deionized Water |
| | 1.0 | Sodium Chloride |
| | 4.0 | Glycerin |

SPF: 14

Heat phase A without TiO₂ to 80° C.;

add TiO₂ and homogenize for 3 min at 11 000 rpm;

heat phase B to 80° C.;

stir phase B into phase A and homogenize;

cool to 40° C. with stirring, then homogenize again.

### EXAMPLE 2

| Phase | % | INCI |
|---|---|---|
| A | 5.0 | Cyclomethicone |
| | 4.0 | Isohexadecane |
| | 1.5 | PEG-7 Hydrogenated Castor Oil |
| | 0.2 | Ceteareth-25 |
| | 3.0 | Titanium Dioxide, Silica Hydrate, Alumina |
| | | Hydrate, Methicone/Dimethicone Copolymer |
| | 0.5 | Cetyl Alcohol |
| B | 1.5 | Acrylamide/Sodium Acryloyldimethyllaurate |
| | | Copolymer Isohexadecane, Polysorbate 80 |
| | 0.5 | Xanthan Gum |
| | ad 100 | Deionized Water |

SPF: 4

Heat phase A without TiO₂ to 80° C.;

add TiO₂ and homogenize for 3 min at 11 000 rpm.

Heat phase B to 80° C. Stir phase B into phase A and homogenize.

Cool to 40° C. with stirring, then homogenize again.

### EXAMPLE 3

| Phase | % | INCI |
|---|---|---|
| A | 3.0 | Octocrylene |
| | 5.0 | Titanium Dioxide, Silica Hydrate, Alumina |
| | | Hydrate, Methicone/Dimethicone Copolymer |
| | 4.0 | C₁₂-C₁₅ Alkyl Benzoate |
| | 8.0 | Ethylhexyl Methoxycinnamate/Diethylamino |
| | | Hydroxybenzoyl Hexyl Benzoate |
| | 6.0 | Caprylic/Capric Triglyceride |
| | 5.0 | PEG-30 Dipolyhydroxystearate |
| | 2.0 | PVP/Hexadecene Copolymer |
| | 2.0 | PEG-45/Dodecyl Glycol Copolymer |
| B | ad 100 | Deionized Water |
| | 0.1 | Disodium EDTA |
| | 1.0 | Magnesium Sulfate |
| | 3.0 | Propylene Glycol |

SPF: 30

Heat phase A without TiO₂ to 80° C.;

add TiO₂ and homogenize for 3 min at 11 000 rpm.

Heat phase B to 80° C. Stir phase B into phase A and homogenize.

Cool to 40° C. with stirring. Homogenize again.

### EXAMPLE 4

| Phase | % | INCI |
|---|---|---|
| A | 8.0 | Dibutyl Adipate |
| | 8.0 | C₁₂-C₁₅ Alkyl Benzoate |
| | 12.0 | Cocoglycerides |
| | 1.0 | Sodium Cetearyl Sulfate |
| | 4.0 | Lauryl Glucoside, Polyglyceryl-2 |
| | | Dipolyhydroxystearate, Glycerin |
| | 2.0 | Cetearyl Alcohol |
| B | 8.0 | Titanium Dioxide, Silica Hydrate, Alumina |
| | | Hydrate, Methicone/Dimethicone Copolymer |
| C | 3.0 | Glycerin |
| | 0.05 | Disodium EDTA |
| | 0.2 | Allantoin |
| | 0.3 | Xanthan Gum |
| | 1.5 | Magnesium Aluminium Silicate |
| | ad 100 | Deionized Water |

SPF: 20

Heat phase A to 80° C.;

mix phase A and B and homogenize for a minimum of 3 min at 11 000 rpm;

heat phase C to 80° C. and homogenize;

mix phase C with phase A/B, homogenize and then cool to 40° C.

### EXAMPLE 5

| Phase | % | INCI |
|---|---|---|
| A | 10.0 | Butylene Glycol |
| | 6.0 | Titanium Dioxide, Silica Hydrate, Alumina |
| | | Hydrate, Methicone/Dimethicone Copolymer |
| B | 8.0 | Caprylic/Capric Triglyceride |
| | 7.5 | Ethylhexyl Methoxycinnamate |
| | 2.0 | Cetearyl Alcohol |
| | 1.5 | Bees Wax |
| | 1.0 | Cetyl Alcohol |
| | 1.0 | Glyceryl Stearate SE |
| | 0.5 | VP/Hexadecene Copolymer |
| | 0.5 | PEG-40 Hydrogenated Castor Oil |
| C | ad 100 | Deionized Water |
| | 5.0 | Glycerin |
| | 0.5 | Xanthan Gum |

SPF: 25

Homogenize phase A;

heat phase B to 80° C.;

mix phase A and B and homogenize for a minimum of 3 min at 11 000 rpm;

heat phase C to 80° C. and homogenize;

mix phase C with phase A/B, homogenize and then cool to 40° C.

### EXAMPLE 6

| Phase | % | INCI |
|---|---|---|
| A | 7.5 | Ethylhexyl Methoxycinnamate |
| | 5.0 | Octocrylene |
| | 3.0 | Cetearyl Glucoside, Cetearyl Alcohol |
| | 5.0 | Titanium Dioxide, Silica Hydrate, Alumina |
| | | Hydrate, Methicone/Dimethicone Copolymer |
| | 2.0 | Glyceryl Stearate, PEG-100 Stearate |
| | 1.0 | Squalene |
| | 0.5 | PEG-7 Hydrogenated Castor Oil |
| | 0.5 | Polysorbate 20 |
| | 2.0 | Aluminum Starch Octenylsuccinate |
| B | 4.0 | Propylene Glycol |
| | 2.0 | Panthenol, Propylene Glycol |
| | 0.1 | Disodium EDTA |
| | 0.2 | Xanthan Gum |
| | 0.5 | Acrylamide/Sodium Acryloyldimethyllaurate |
| | | Copolymer, Isohexadecane, Polysorbate 80 |
| | ad 100 | Deionized Water |

Heat phase A without TiO₂ to 80° C.;

add TiO₂ and homogenize for 3 min at 11 000 rpm.

Heat phase B to 80° C. Stir phase B into phase A and homogenize.

Cool to 40° C. with stirring. Homogenize again.

Example 7

| Phase | % | INCI |
|---|---|---|
| A | 10.0 | Ethylhexyl Methoxycinnamate/Diethylamino |
| | | Hydroxybenzoyl Hexyl Benzoate |
| | 2.5 | Di-C₁₂-C₁₃ Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C₁₂-C₁₃ Alkyl Malate |
| | 3.0 | Titanium Dioxide, Silica Hydrate, Alumina |
| | | Hydrate, Methicone/Dimethicone Copolymer |
| C | ad 100 | Deionized Water |
| | 5.0 | Glycerin |
| | 0.5 | Xanthan Gum |
| | 1.0 | Sodium Cetearyl Sulfate |

SPF: 28

Heat phase A to 80° C. and homogenize;

heat phase B to 80° C.;

mix phase A and B and homogenize for a minimum of 3 min at 11 000 rpm;

heat phase C to 80° C. and homogenize;

mix phase C with phase A/B, homogenize and then cool to 40° C.

### EXAMPLE 8

| Phase | % | INCI |
|---|---|---|
| A | 3.0 | Isopropyl myristate |
| | 8.0 | Simmondsia Chinensis (Jojoba Seed Oil) |
| | 4.0 | Ethylhexyl Methoxycinnamate |
| | 4.2 | Titanium Dioxide, Silica Hydrate, Alumina |
| | | Hydrate, Methicone/Dimethicone Copolymer |
| | 1.0 | Dimethicone |
| | 5.0 | Zinc Oxide, Triethoxycaprylylsilane |
| | 6.0 | PEG-7 Hydrogenated Castor Oil |
| | 2.0 | PEG-45/Dodecyl Glycol Copolymer |
| B | ad 100 | Deionized Water |
| | 0.1 | Disodium EDTA |
| | 5.0 | Glycerin |
| | 1.0 | Magnesium Sulfate |

SPF: 15

Heat phase A without TiO₂ to 80° C.;

add TiO₂ and homogenize for 3 min at 11 000 rpm.

Heat phase B to 80° C. Stir phase B into phase A and homogenize.

Cool to 40° C. with stirring. Homogenize again.

What is claimed is:

1. A cosmetic or dermatological preparation for protecting human skin and hair against UV radiation, comprising surface-coated titanium dioxide particles which are transparent in the visible region and have a crystallite size of from 10 to 20 nm and a specific surface area of from 90 to 110 m²/g, wherein the surface coating of the titanium dioxide particles comprises a multicoating of a) aluminum oxide and b) methicone or a copolymer of methicone and dimethicone.

2. The cosmetic or dermatological preparation according to claim 1, wherein the surface coating of the titanium dioxide particles comprises silicon dioxide as additional component c).

3. The cosmetic or dermatological preparation according to claim 1 with a content of surface-coated titanium dioxide particles which are transparent in the visible region in the range from 0.1 to 25% by weight, based on the total weight of the cosmetic or dermatological preparation.

4. The cosmetic or dermatological preparation according to claim 2, wherein the titanium dioxide particles are coated by
a) a first layer of aluminum oxide or a mixture of aluminum oxide and silicon dioxide and
b) an outer layer of a copolymer of methicone and dimethicone.

5. The cosmetic or dermatological preparation according to claim 1, wherein the transparent surface-coated titanium dioxide particles have a titanium dioxide content of from 70 to 92% by weight.

6. The cosmetic or dermatological preparation according to claim 2, wherein the transparent surface-coated titanium dioxide particles have a silicon dioxide content of from 4 to 10% by weight.

7. The cosmetic or dermatological preparation according to claim 1, wherein the transparent surface-coated titanium dioxide particles have an aluminum oxide content of from 1 to 10% by weight.

8. The cosmetic or dermatological preparation according to claim 1, wherein the transparent surface-coated titanium dioxide particles have a methicone or methicone/dimethicone copolymer content of from 3 to 10% by weight.

9. The cosmetic or dermatological preparation according to claim 1, wherein the titanium dioxide particles have a rutile fraction of from 94 to 100%.

10. The cosmetic or dermatological preparation according to claim 1, which comprises the transparent surface-coated titanium dioxide particles alone or together with other absorbing compounds known for cosmetic and dermatological preparations.

11. The cosmetic or dermatological preparation according to claim 2 with a content of surface-coated titanium dioxide particles which are transparent in the visible region in the range from 0.1 to 25% by weight, based on the total weight of the cosmetic or dermatological preparation.

12. The cosmetic or dermatological preparation according to claim 3, wherein the titanium dioxide particles are coated by
a) a first layer of aluminum oxide or a mixture of aluminum oxide and silicon dioxide and
b) an outer layer of a copolymer of methicone and dimethicone.

13. The cosmetic or dermatological preparation according to claim 2, wherein the transparent surface-coated titanium dioxide particles have a titanium dioxide content of from 70 to 92% by weight.

14. The cosmetic or dermatological preparation according to claim 3, wherein the transparent surface-coated titanium dioxide particles have a titanium dioxide content of from 70 to 92% by weight.

15. The cosmetic or dermatological preparation according to claim 3, wherein the transparent surface-coated titanium dioxide particles have a silicon dioxide content of from 4 to 10% by weight.

16. The cosmetic or dermatological preparation according to claim 2, wherein the transparent surface-coated titanium dioxide particles have an aluminum oxide content of from 1 to 10% by weight.

17. The cosmetic or dermatological preparation according to claim 4, wherein the transparent surface-coated titanium dioxide particles have a titanium dioxide content of from 70 to 92% by weight.

18. The cosmetic or dermatological preparation according to claim 5, wherein the transparent surface-coated titanium dioxide particles have a titanium dioxide content of from 70 to 92% by weight.

19. The cosmetic or dermatological preparation according to claim 3, wherein the transparent surface-coated titanium dioxide particles have an aluminum oxide content of from 1 to 10% by weight.

20. The cosmetic or dermatological preparation according to claim 4, wherein the transparent surface-coated titanium dioxide particles have a titanium dioxide content of from 70 to 92% by weight.
